(19) United States
(12) Patent Application Publication
(10) Pub. No.: US 2003/0195539 A1
(43) Pub. Date: Oct. 16, 2003

(54) Device for removable connection of a functional unit to a housing

(76) Inventors: Jurg Attinger, Stein am Rhein (CH); Werner Maag, Glarus (CH); Lorenz Egli, Neuhausen am Rheinfall (CH)

Correspondence Address: Alcon Research, Ltd., Jeffrey S. Schira, Q-148, 6201 South Freeway, Fort Worth, TX, 76134-2099, US

(21) Appl. No.: 10/411,540

(22) Filed: Apr. 10, 2003

(30) Foreign Application Priority Data
Apr. 16, 2002 (CH) 0634/02
Dec. 2, 2002 (CH) 2031/02

(51) Int. Cl. A61B 17/32

(52) U.S. Cl. 606/170

(57) ABSTRACT

A device for removably connecting an ophthalmologic functional unit to a housing is configured as a hand piece and includes a connecting member disposed at the housing with a cylindrical part integrally formed thereon for receiving the functional unit with a surgical tool attached for the ophthalmologic procedures, wherein the housing including certain elements is configured as a first subassembly and the functional unit with the surgical tool attached is configured as a second subassembly and the two units are form-fittingly connected to each other and wherein an axially movable first rod supported in the first unit is operatively engaged with a sliding pin in the second unit to thereby actuate the surgical tool.

### CROSS-REFERENCES TO RELATED APPLICATIONS

This application claims the priority of Swiss Patent Applications Serial No. CH 2002 0634/02, filed Apr. 16, 2002, and Serial No. CH 2002 2031/02, filed Dec. 2, 2002, pursuant to 35 U.S.C. 119(a)-(d), the disclosure of which is incorporated herein by reference.

### BACKGROUND OF THE INVENTION

The present invention relates, in general, to instruments used in ophthalmologic surgery, and more particularly relates to an instrument including a device for removable connection of a functional unit with a surgical tool for ophthalmologic procedures, to a housing and operable by manual means or motor means.

Instruments that are suitably configured for ophthalmologic surgery are generally known. For example, instruments known in the prior art comprise a housing, which is configured as a hand piece and provided with drive elements for manual or motor operation and a functional unit to which the surgical tool is connected, which tool is at a distal end thereof and configured as a cutting-, grasping- or clamping element. In assembled condition, the surgical tool for carrying out the surgical procedures is connected to the functional unit and is operated by means of drive elements provided in the housing.

It would be desirable and advantageous to provide an improved surgical instrument, which obviates prior art shortcomings.

### SUMMARY OF THE INVENTION

According to one aspect of the present invention, a surgical instrument is provided which includes a functional unit for ophthalmologic purposes which is provided with a surgical tool and which can be removably connected to a housing without auxiliary attachments. With the instrument according to the present invention a secure handling of the tool as well as the ability to carry out exact movements and specifically a direct transmission of movements for the actuation of the tool is realized without the need for auxiliary means.

In accordance with one aspect of the present invention, the surgical instrument includes a housing, a connecting member disposed at the housing and an adapter disposed at the connecting member for receiving the functional unit which is movably guided in a groove- or slot-shaped track provided at the connecting member or the functional unit, and when brought into an axial terminal position or, in reverse, by a rotary motion, about the common longitudinal axis, is held in a form-fit manner at the adapter in such a manner so as to be operatively engaged with a rod axially supported in the connecting member and engageable with at least one drive and movable in axial direction for operative engagement with a sliding pin, which sliding pin is disposed in the functional unit and connected to the surgical tool to thereby actuate the tool.

Further advantages and other features of the instrument according to the present invention are described in the following description and in connection with the accompanying drawings.

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1 is a perspective view of a first embodiment of the surgical instrument according to the present invention including a housing configured as a hand piece for removable disposition of a functional unit at the housing;

FIG. 2 is a front view of the surgical instrument according to FIG. 1 showing the functional unit detached with respect to the housing;

FIG. 3 is a section of the front portion of the surgical instrument according to FIG. 1 or FIG. 2 on an enlarged scale with the functional unit partially disposed at the connecting member;

FIG. 4 is a second embodiment of the surgical instrument with the functional unit in detached position;

FIG. 5 is a partial section of a first variation of the embodiment according to FIG. 4 of the connecting member for the surgical instrument;

FIG. 6 is a side view of the connecting member according to FIG. 5;

FIG. 7 is a partial section of the embodiment of the surgical instrument shown in FIG. 4 with the functional unit shown in detached position;

FIG. 8 is a partial section of a second variation of the connecting member of the surgical instrument according to FIG. 7;

FIG. 9 is a side view of the connecting member according to FIG. 8;

FIG. 10 is a perspective view of a third embodiment of the surgical instrument with a functional unit disposed thereon;

FIG. 11 is a front view of the surgical instrument according to FIG. 10 with the functional unit shown in detached position;

FIG. 12 is a partial section of a front portion of the surgical instrument according to FIG. 10 and FIG. 11 on an enlarged scale with a functional unit disposed at the connecting member;

FIG. 13 is partial section of the connecting member for the instrument according to FIG. 10 to FIG. 12;

FIG. 14 is a section along line A-A of FIG. 13 of the connecting member according to FIG. 13;

FIG. 15 is a perspective view of a fourth embodiment of the surgical instrument according to the present invention with the functional unit disposed thereon;

FIG. 16 is a section of a partial portion to the surgical instrument according to FIG. 15 on an enlarged scale showing the functional unit in detached position;

FIG. 17 is a section of the functional unit seen along line B-B of FIG. 16;

FIG. 18 is a partial view of a fifth embodiment of the surgical instrument with the functional unit shown in detached position;

FIG. 19 is a further embodiment of the surgical instrument with the housing and an intermediary member, and the functional unit shown in detached position;

FIG. 20 is a first example of the functional unit with an intermediary member screwed-in for the surgical instrument according to FIG. 19;

FIG. 21 is a second example of showing the adapter slipped onto the connecting member for the detachable connection of the functional unit at the housing;

FIG. 22 is the surgical instrument according to FIG. 19 showing each of the elements in assembled position;

FIG. 23 is a perspective view of a portion of the connecting member for each of the instruments as shown in FIG. 1 to FIG. 22;

FIG. 24 is a front view of the connecting member according to FIG. 23 with a guide track arranged therein; and

FIG. 25 is a developed view of the guide track in the connecting member shown on an enlarged scale.

### DETAILED DESCRIPTION OF PREFERRED EMBODIMENTS

Throughout all the Figures, same or corresponding elements are generally indicated by same reference numerals. These depicted embodiments are to be understood as illustrative of the invention and not as limiting in any way.

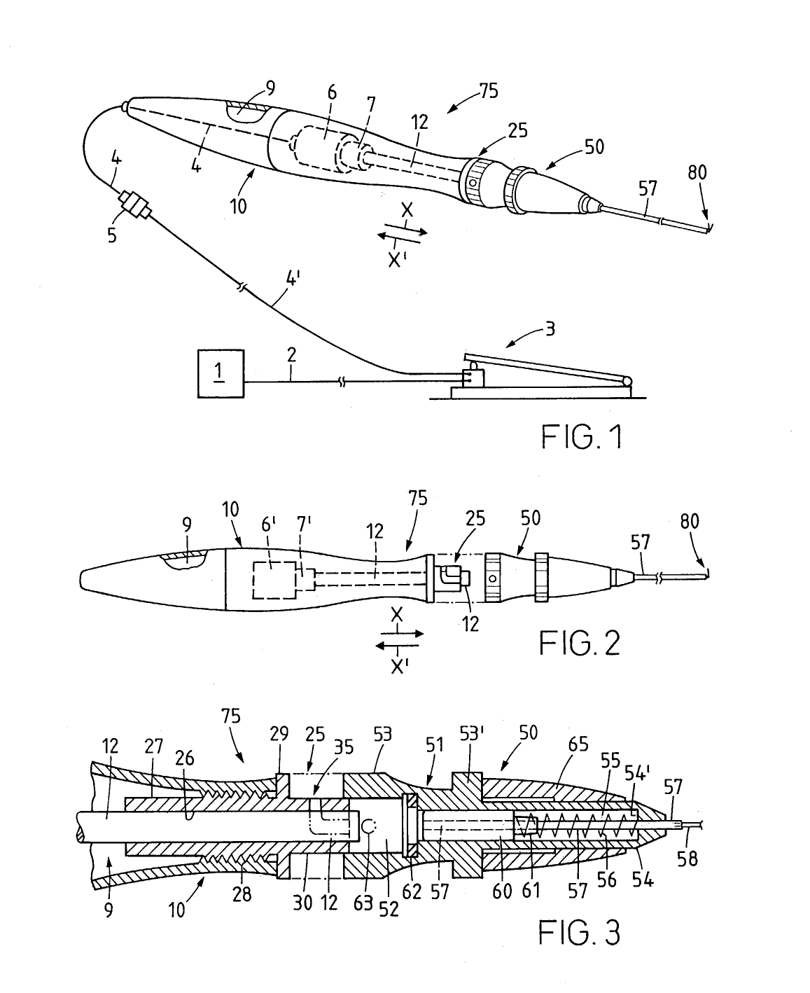

For a general illustration of the present invention, FIG. 1 through FIG. 25 show various configurations and variations of embodiments of the surgical instrument. Each of the instruments essentially comprise a housing 10 configured as a hand piece and a functional unit 50. Supported in housing 10 is a first rod 12, which is operatively connected with manual or motor means for movement in axial direction. The first rod 12, in operative engagement with a sliding pin 60 for moving a tube shaped probe 57 disposed in the functional unit 50 in axial direction relative to a second rod 58, which is co-axially supported in the probe 57. When moving the first rod 12 in conjunction with operatively connected locking peg 63, a probe disposed at the functional unit 50 is moved in axial direction relative to a co-axially-arranged second rod 58. Due to the axial relative motion of the probe with respect to the second rod 58, tool 80 connected to the distal end thereof, the instrument can be actuated for operation.

It is pointed out that the cutting-, grasping- or clamping-element shown schematically in the Figures are hereinafter designated generally as tool 80. The functional unit 50 as shown in the various figures in either an attached or detached position are configured as the functional unit described in connection with the functional unit 50 shown in FIG. 3.

Turning now to the drawing, and in particular to FIG. 1, there is shown a perspective view of a first embodiment of the surgical instrument generally designated with 75. The instrument 75 is configured for carrying out ophthalmologic surgery and comprises a housing 10 configured as a hand piece and a connecting member 25 disposed at the housing and the functional unit 50 which is removably attached to the housing 10. At the front end of the functional unit 50, a probe 57 which is configured as a hollow needle is attached and supported therein is the second rod 58 with the tool 80 disposed at the distal end thereof (FIG. 3). Due to the relative axial movement of the probe with respect to the second rod 58, the tool 80 can be actuated.

As shown in the first embodiment in FIG. 1, a drive 6 is disposed inside the interior 9 of the housing 10 for actuating tool 80. At one of its ends, drive 6 is operatively connected via a cam 7 with the first rod 12, which is slidable in axial direction according to arrow X or X′. At its other end, drive 6 is connected to switch 3 via a line, for example a first line 4 and 4′ with cam 5 in an inline arrangement. The switch 3, which may be configured as a foot pedal, is connected to a power source 1 via a second line 2.

The first rod 12 disposed in the housing 10 and operatively connected to drive 6 can be moved in axial direction by drive 6 through pneumatic, hydraulic or electro-motor means. In a first variation, drive 6 is operatively connected with a pressure generating power source 1 for pneumatic activation. In the second variation, the drive is configured as an electro-motor 6 and hooked up to an electrical source for operation. In a further variation, the drive is configured as a hydraulically activated drive 6 and operatively connected to a power source for generating hydraulic pressure. Each of the various drives is preferably activated through the switch 3 and foot pedal.

Shown in front view in FIG. 2 is a first variation of instrument 75 which includes the housing 10 and the connecting member 25 disposed therein and the functional unit 50 shown at some distance and detached from the housing. As compared to the instrument 75 in FIG. 1, here, the interior 9 of the housing 10 for example carries a removable battery 6′. The battery 6′ is operatively connected via a cam 7′ to the first rod 12 which is axially slidable according to arrow X or X′.

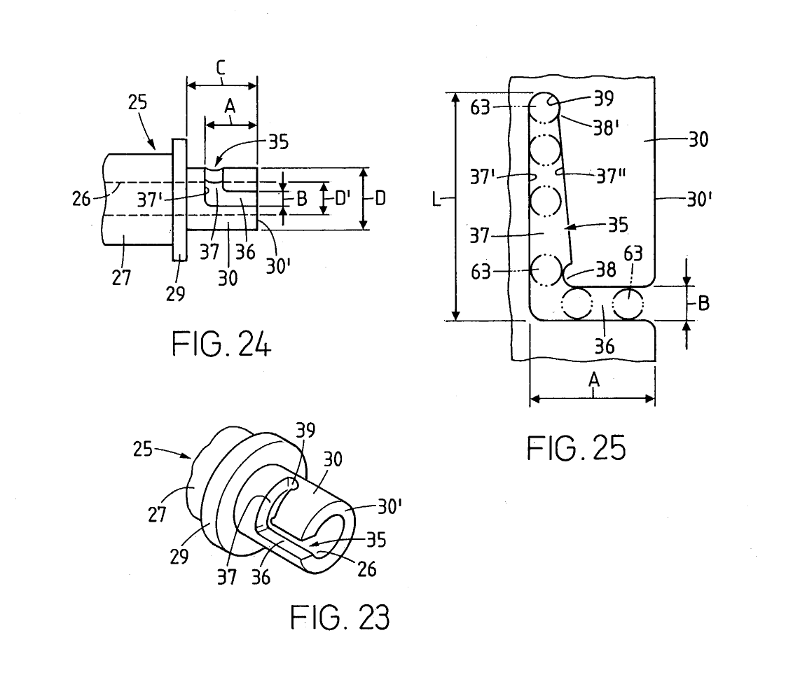

In FIG. 3, the section of the front portion of instrument 75 according to FIG. 1 or FIG. 2 is shown on an enlarged scale with a portion of the housing 10 and the functional unit 50 in partially detached position. The connecting member 25 with the co-axially supported first rod 12 is disposed in the interior 9 of housing 10. The connecting member 25 is provided with a through-bore 26 in axial direction for supporting the first rod 12 in a guide sleeve 27, and provided with a partial thread 28 and a flange 29 in addition to an adapter 30. The connecting member 25 is screwed by means of the thread section 28 into an inner thread provided in the front section of housing 10 and not shown here in detail. The cylindrical adapter 30 integrally formed with flange 29 is provided with a guide track 35 for receiving and form-fittingly connecting with locking peg 63 provided at the functional unit 50. The guide track 35 which is configured for receiving the locking peg 63 will be described infra in connection with FIG. 23 to FIG. 25.

The functional unit 50 shown in section in FIG. 3 comprises a housing 51 with integrally formed handle portions 53 and 53′ and a guide sleeve 54 integrally formed in axial direction. The guide sleeve 54 is preferably provided with a cap 65 attached with means not shown here in detail. The housing 51 is provided with a first recess 52 for sliding onto the adapter 30 and a seal 62 disposed therein and second recess 55. Disposed in the second recess 55 is the sliding pin 60 operatively connected to the tube-shaped probe 57 and a pressure spring 56. The second rod 58 provided with tool 80 (FIGS. 1 and 2) is attached to the housing 51 in a manner not shown here in detail so that the sliding pin 60 together with the tube-shaped probe 57 is axially moved relative to the second rod 58 to thereby actuate the tool 80. One end of the pressure spring 56 is disposed at a shoulder 61 of the sliding pin 60 and with the other end bears upon the inner wall 54′ of the guide sleeve 54.

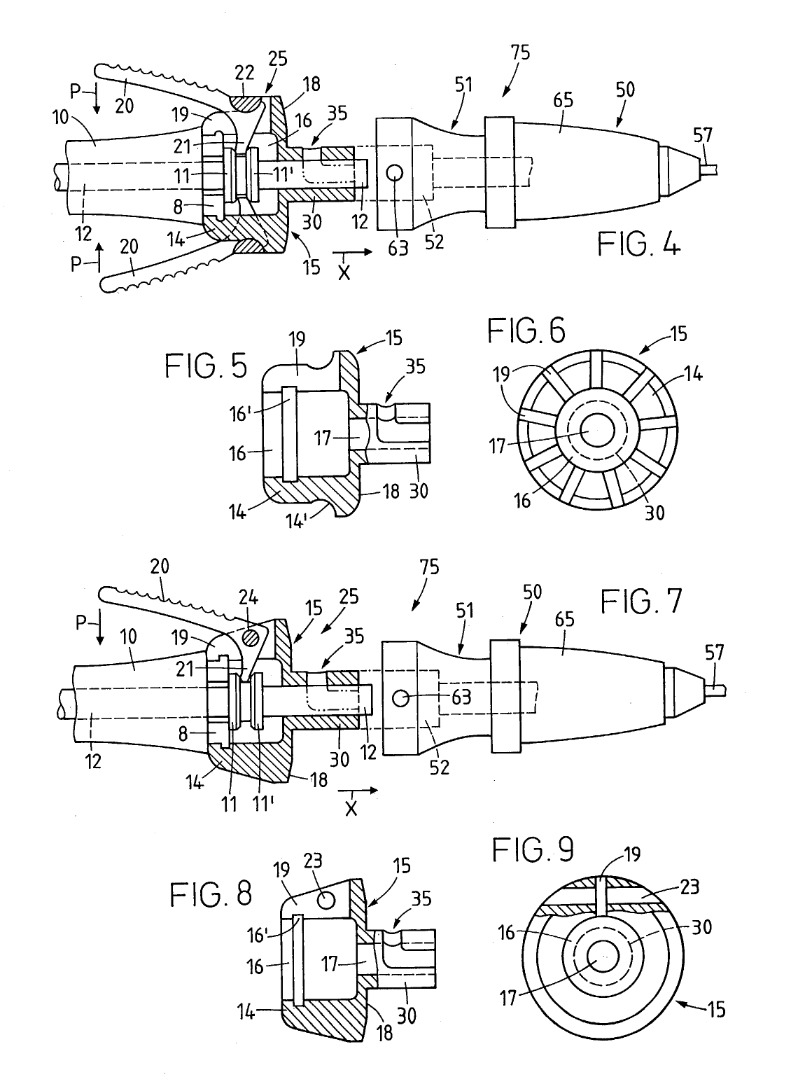

In FIG. 4, the second embodiment is shown including a manually operated instrument 75 with a portion of the housing 10 and the connecting member 25 disposed thereon, in addition to the functional unit 50 shown in detached position. As compared to the instrument according to FIG. 1 and FIG. 2, here the connecting member 25 disposed at the front end of the housing 10 is provided with a headpiece 15. Disposed at the headpiece 15 is the housing 10, which is provided with two or more catch members 8 circumferentially placed at the housing 10. Furthermore, at least two levers 20 are supported at head piece 15 and held by a ring 22 or similar, which levers 20 are operatively connected to the first rod 12 by means of a catch 21. The ring 22, which has an approximately semicircular, profiled cross section and is made for example from elastomeric material. In the embodiment as shown, the two levers 20 with the catch 21 are disposed between two circular retaining pieces 11 and 11′ at the first rod 12 in axial distance to each other. When activating the two levers 20 in direction of arrow P, the first rod 12 moves axially in direction of arrow X. The axial movement of the first rod 12 causes the engagement with sliding pin 60 as in FIG. 3 when the functional unit 50 is in attached position to thereby actuate the surgical tool 80, as in FIGS. 1 and 2.

In FIGS. 5 and 6, the head piece 15 for the connecting member 25 in accordance with FIG. 4 is shown in a sectional side view and includes a support piece 14 having a wall 18 and provided with a recess 16 and a circular groove 16′. Disposed at the wall 18 is the cylindrical adapter 30 provided with an axial bore 17 and the guide track 35. The support piece 14 is provided with a circular recess 14′ for the ring 22 with the semicircular profiled cross section (FIG. 4) as well as at least two, preferably several slots 19 (FIG. 6) disposed circumferentially and each configured for receiving one of the levers 20 (FIG. 4).

Another variation of the manually operable instrument 75 is shown in FIG. 7 according to FIG. 4 showing a portion of the housing 10 and disposed at the housing 10 the connecting member 25 and the functional unit 50 in a detached position. As compared to the instrument as shown in FIG. 4, here, the connecting member 25 disposed at the front end of the housing 10 is provided with a single lever 20 supported in the slot 19 at support piece 14 of head piece 15. The housing 10 is likewise attached to the head piece 15 with two or more circumferentially disposed catch members 8. In the embodiment as seen in FIG. 7, lever 20 is supported at a pin 24 disposed in head piece 15 and connected by means of the catch 21 between the two circular retaining pieces 11 and 11′ arranged axially at a distance from each other. When actuating the lever 20 in direction of arrow P, the first rod 12 slides in direction of arrow X and the attached functional until 50 is brought into operative engagement with the sliding pin 60 (FIG. 3).

FIGS. 8 and 9 shows the head piece 15 for the connecting member 25 in accordance with the side view respectively the sectional view in FIG. 7. The head piece 15 is configured essentially analog the head piece 15 in accordance with FIG. 5 and FIG. 6 and comprises support piece 14 with wall 18 in addition to the cylindrical adapter 30 which is disposed in the guide track 35. In a variation to the head piece 15 as shown in FIG. 5 and FIG. 6, in the present example, the head piece 15 is provided with only one slot 19 according to FIG. 8 and FIG. 9 and a transversely oriented bore 23 for receiving pin 24. Lever 20, operatively connected to the first rod 12, is pivotably disposed at the pin 24.

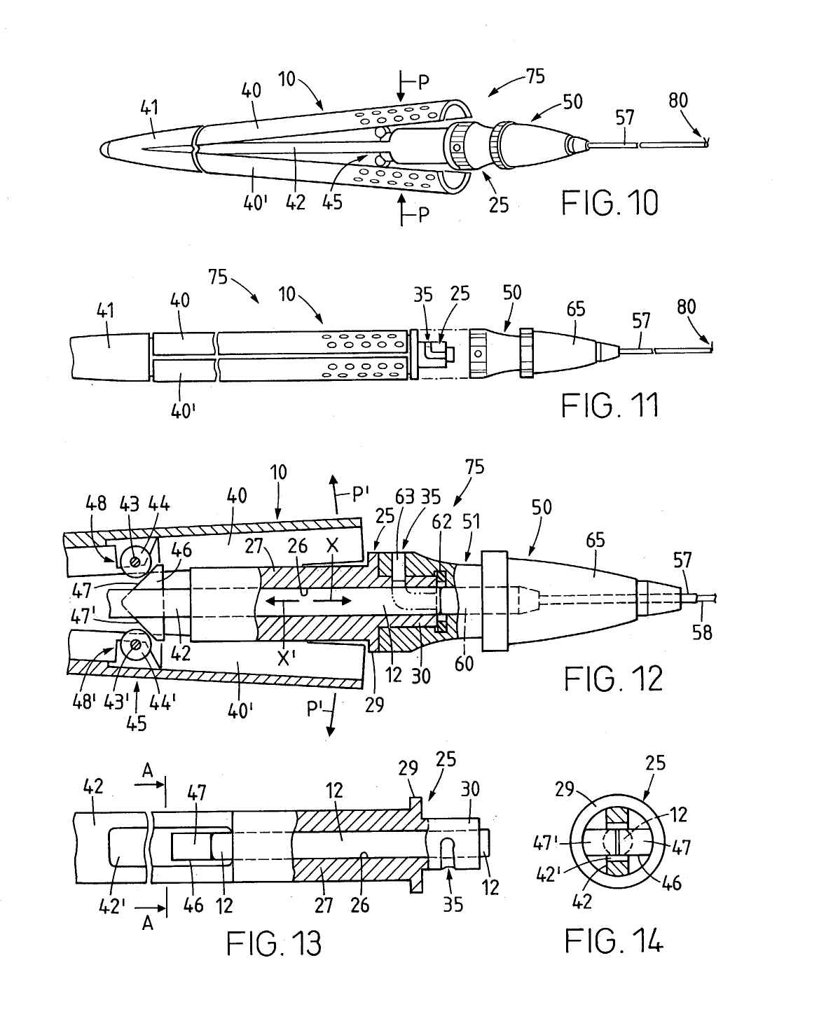

A perspective view of a third embodiment is shown in FIG. 10 and in FIG. 11 the front view if the manually operable instrument 75 is shown. The instrument 75 includes the housing 10, the connecting member 25 and the functional unit 50 with the tool 80 disposed at the distal end thereof. As shown in FIG. 10, the housing forms a generally cylindrical handpiece and is split longitudinally into two elongate housing parts 40 and 40′ of substantially semicircular configuration with a closure cap 41 disposed at one end. A carrying arm 42 is disposed between the two housing parts 40 and 40′ and connected with the housing parts 40 and 40′ in a manner not shown here in detail. An actuating mechanism 45 as well as the connecting member 25 attached at the carrying arm 42 are disposed in the front area between the two housing parts 40 and 40′. When the functional unit 50 is detached, the two housing parts 40 and 40′ are automatically closed due to their own spring elastic restoring force as shown in FIG. 11.

In FIG. 12 are a partial section view of the front portion of the instrument 75 in accordance with FIG. 10 is shown on an enlarged scale and a partial view of the housing 10 with the housing parts 40 and 40′ as well as the operatively connected actuating mechanism 45. Further shown is the connecting member 25 with the functional unit 50 disposed at the carrying arm 42. In this position, the first rod 12 is pressed against the sealing ring 62 in a sealing manner. The actuating mechanism 45 comprises a support 48 and 48′ disposed at the inside of each of the housing parts 40 and 40′. Each of the supports 48 and 48′ is configured for receiving a support roller 44 and 44′ supported at an axle 43 and 43′. The connecting member 25 comprises the guide sleeve 27 provided with an axial through-bore 26 and a flange 29 with the integrally formed cylindrical adapter 30. Disposed in guide sleeve 27 is first rod 12, which, as shown in the example of the embodiment here, is provided at one end with the wedge-shaped sliding member 46.

When moving the two housing parts 40 and 40′ by manual operation in the direction of arrow P (FIG. 10) or P′ (FIG. 12), the two sliding rollers 44 and 44′ slide along the sloped gliding surfaces 47 and 47′ of sliding member 46. When the two housing parts 40 and 40′ are moving in the direction of arrow P′ (FIG. 12), the first rod 12 is moved in direction of arrow X′ and when moving in direction of arrow P, is moving into direction of arrow X.

FIG. 13 is a top view of a section of the carrying arm 42 disposed at the front end of connecting member 25, showing the guide sleeve 27 and flange 29 with the integrally formed adapter 30 with guide track 35. The first rod 12 with the sliding member 46 disposed thereon is supported in the through-bore 26 and guided in the recess 42′ formed in longitudinal direction in carrying arm 42. FIG. 14 shows a section of the carrying arm 42 in accordance with line A-A of FIG. 13 and the sliding member 46 disposed at the rod 12 under controlled guidance in recess 42′ in addition to flange 29 of the connecting piece 25.

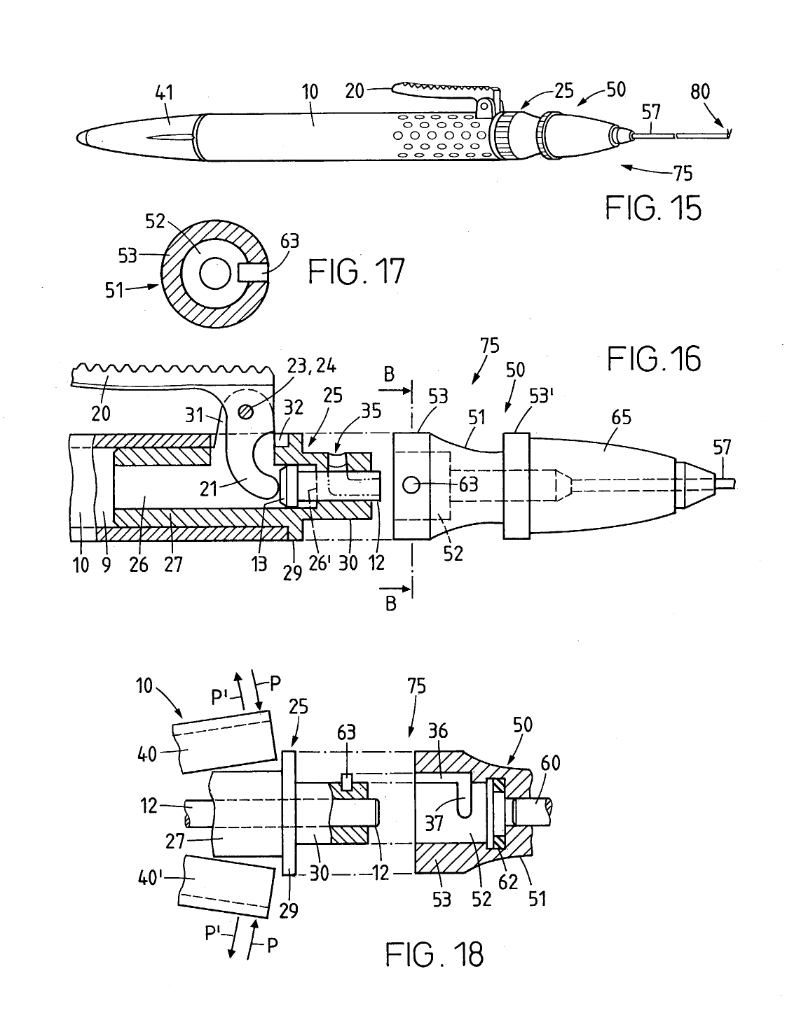

FIG. 15 shows a perspective view of a fourth embodiment of the manually operable instrument 75 according to the present invention. The instrument comprises the housing 10 provided with the closure cap 41 and the connection piece 25 for the functional unit 50 and the tool 80 disposed at the distal end.

FIG. 16 shows a partial section of the front section of the instrument 75 on an enlarged scale in accordance with FIG. 15, wherein a section of the housing 10 is shown in addition to the connecting member 25 disposed at the housing and the first rod 12 co-axially supported in the connecting member 25. FIG. 16 shows a front view of the functional unit 50 in detached position. The functional unit 50 including the various elements is configured in accordance with the functional unit 50 as described in connection with FIG. 3. The connecting member 25, which is provided with the axial through-bore 26 for supporting the first rod 12 is attached at the interior 9 of housing 10 by means not shown here in detail, for example, through an adhesive of welding connection. The connecting member 25 comprises the guide sleeve 27 with the flange 29 and the cylindrical adapter 30 with guide track 35 integrally formed thereon.

In the variation as shown in FIG. 16, at least one support member 31, preferably two support members 31, each provided with a bore 23 in parallel distance to each other are placed circumferentially at the outer diameter of the guide sleeve 27. Between the two support members 31, lever 20 provided with the catch 21 is supported at the pin 24 in such a manner that the catch 21 is in operative connection with a head piece 13 integrally formed with rod 12. The axial movement of the first rod 12 caused by the lever 20, respectively the catch 21, is limited by the stop 26′, which is provided in the interior of through-bore 26. The housing 10 is provided with an axially slot-shaped recess 32 for insertion of the connecting member 25 which is provided with one or two support members 31.

FIG. 17 shows a section of the functional unit 50 with the housing part 51 along line B-B as shown in FIG. 16. Further shown is the circular ring-shaped section 53 and the locking peg 63. One end of the locking peg 63 extends into the interior 52 of the housing part 51 and the other end is attached in the wall of the section 53 by means not shown here in detail.

FIG. 18 shows a section of a fifth embodiment of the instrument 75 according to the present invention, wherein the guide track 35 disposed in the recess 52 of housing part 51 of the functional unit 50. The guide track 35 is provided with the an axial groove 36 and a circumferentially oriented second groove 37. In this example of the embodiment, the locking peg 63 provided for insertion into the guide track 35 is disposed at the cylindrical adapter 30 of connecting member 25 provided with flange 29 and attached by means not shown here in detail.

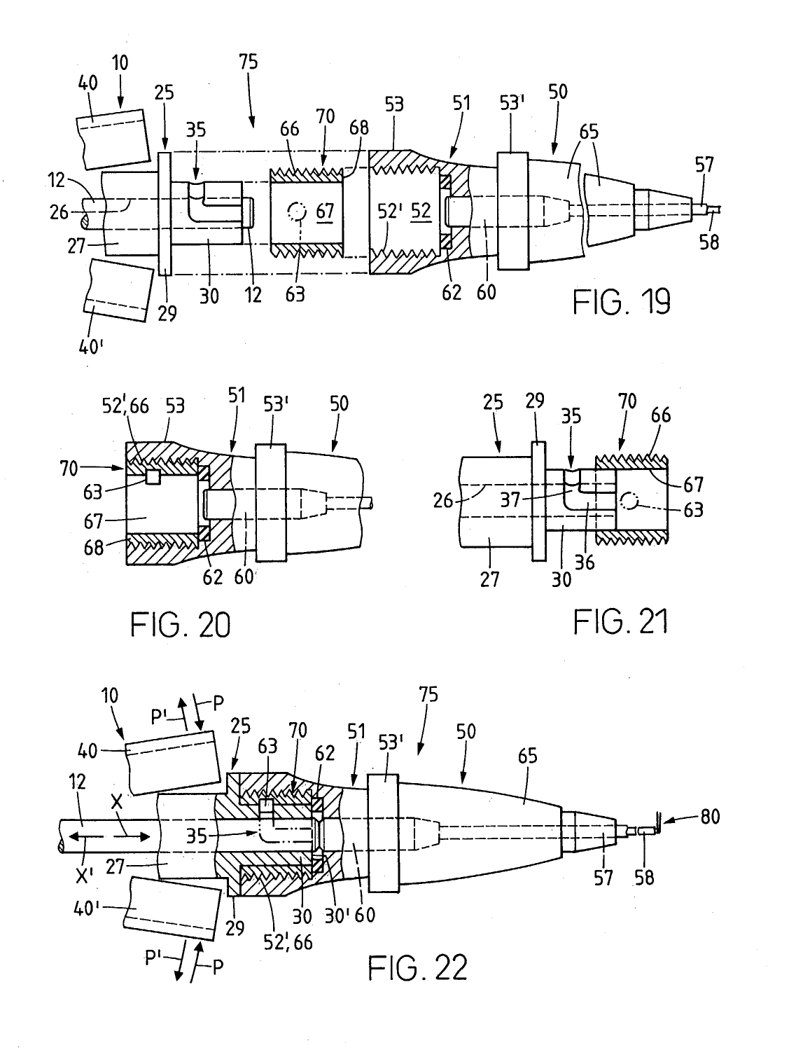

FIG. 19 shows an exploded view of a further embodiment of the instrument 75 according to the present invention showing the housing 10 with connecting member 25 and an intermediary member 70 which is provided with an exterior thread 66 for a detachable connection of the functional unit 50 with the housing 10 formed as a hand piece. The housing 10 includes the two housing parts 40 and 40′ configured similar to the housing 10 as shown in FIG. 10 to FIG. 12. Disposed between the two housing parts 40 and 40′ is the connecting member 25 which is provided with the guide sleeve 27, the bearing flange 29 and the cylindrical adapter 30. The first rod 12 which is axially movable by means of the actuating mechanism 45 as shown in FIG. 12, is disposed in the guide sleeve 27 respectively in bore 26. Further shown is the guide track 35 at the cylindrical adapter 30 of the connecting member 25, which is further described in connection with FIG. 23 to FIG. 25.

A partial sectional view of the functional unit 50 is shown in FIG. 19 and comprises the housing 51 with the cap 65 and the seal 62 supported in recess 52. In a variation of the afore-described functional unit's 50, in the functional unit 50 as shown in FIG. 19 the recess 52 is provided with an interior thread 52′. The recess 52 provided with the thread 52′, is configured for a threaded connection of a intermediate member 70. Furthermore, it is shown that the axial sliding pin 60 is operably connected with the tube-shaped probe 57. The second rod 58 is disposed in tube-shaped probe 57, which, at the distal end is provided with the surgical tool not shown here. For operation of the surgical tool the sliding pin 60 is axially movable in conjunction with the tube-shaped probe 57 relative to the second rod 58.

FIG. 19 shows a section of the intermediary member 70 configured as a cylindrical sleeve body and is provided with the exterior thread 66 and an axial through-bore 67. Disposed in the wall 68 of intermediary member 70 is the peg 63, which extends partially into the through-bore 67 and is attached by means not shown there in detail.

As shown in the embodiment of FIG. 20, the intermediary member 70 is screwed into the interior thread 52′ of functional unit 50. The two parts 50 and 70 are forming thereby a unit, which when attaching the instrument to the housing 10 slides unto the cylindrical adapter 30 of the connecting member 25. When the functional unit 50 moves in axial direction, the peg 63 is pushed up to a stop 37′ (FIG. 24) in the guide track 35 and in this end position turned together with the screwed-in intermediary member 70 relative to the connecting member 25 about the longitudinal axis until the peg 63 form-fittingly locks into a recess 39 (FIG. 25) of guide track 35.

In the example as shown in FIG. 21 the intermediary member 70 has been axially slipped onto the cylindrical adapter 30 of connecting member 25 until the peg 63 bears on the stop 37′ of guide track 35. In the end position, the intermediary member 70 is turned relative to the cylindrical adapter 30 of the connecting member 25 about the longitudinal axis (no shown here) until the peg 63 form-fittingly locks into the recess 39 (FIG. 25). In the end position, the connecting member 25 disposed at the first housing 10 and the intermediary member 70 form a unit. In this embodiment, the functional 50 provided with the interior thread 52′ is screwed onto the outer thread 66 provided at the intermediary member 70.

FIG. 22 shows a partial section of the instrument 75 as afore-described in connection with FIG. 19 to FIG. 21 in an assembled condition comprising the partially shown housing 10 with the two housing parts 40 and 40′, movable relative to each other and the connecting member 25 and the intermediary member 70 with the screwed-on functional unit 50. In this position, the peg 63 is in operative connection with the guide track 35 provided at the cylindrical adapter 30 of the connecting member 25 and the first rod 12 with the sliding pin 60 of functional unit 50 for actuating the surgical tool 80. In this position, the front side 30′ of adapter 30 is sealingly pressed against the sealing ring 62 of functional unit 50.

Due to a forward turning motion of housing 10 about the longitudinal axis under a correspondingly arced angle relative to the functional unit 50 (or in reverse), a form-fitting and stable connection of each of the elements is realized and resulting in a secure handling of the afore-described instrument 75 as shown in the drawings.

When applying the manual force to the two housing parts 40 and 40′ by means of the actuating mechanism 45 disposed in housing 10 in accordance with the direction of arrow P, the first rod 12 with the sliding pin 60 is moved in direction of arrow X thereby activating the tool 80 connected to the functional unit 50 for the surgical procedure. When the two housing parts 40 and 40′ are moving in the direction of arrow P′, the first rod 12 is moved in direction of arrow X′.

FIG. 23 shows a perspective view of the section of the connecting member 25 with the flange 29 and the integrally formed cylindrical adapter with the through-bore 26 and provided with the guide track 35. Starting from the front-end 30′ of the adapter 30, the guide track 35 is provided with an axially formed first groove 36 and approximately transverse thereto the circumferentially oriented second groove 37. The second groove 37 is bordered by the semicircular recess 39 approximately shaped in correspondence with peg 63.

A front view of a preferred embodiment shown in FIG. 24 comprises the connecting member 25 and the cylindrical adapter 30 provided with guide track 35. The adapter 30 starting from flange 29 and extending to front side 30′ has a length C of 6 mm and an outer diameter D of 4.5 mm. The axial bore 26 in connecting member 25 has a diameter D′ of 2.4 mm. The first groove 36 starting from the front side 30′ in a distance A of 4.5 mm to the stoop 37′ has a width B of 1.5 mm.

FIG. 25 shows a developing view of the adapter 30 with guide track 35 and starting from the front side 30′ in axial direction the first groove 36 and approximately transverse thereto the second groove 37. The second groove 37 is bordered by the approximately semi-circular-shaped recess 39, which is dimensioned in correspondence to locking peg 63. Provided in transition from the first groove 36 to the second groove 37 and oriented in direction of the second groove, is a convex, arc-shaped first locking cam 38 and at the end of the second groove 37, a convex, arc-shaped second locking cam 38′ and associated with the recess 39.

In FIG. 25, the several phases of insertion of the locking peg 63 into the guide track 35 are shown in a schematic representation. During the first phase, the locking peg 63 slides alongside the first groove 36 in the direction of the second groove 37. As soon as the locking peg 63 pushes against the one inner wall 37′ of the second groove 37, a motion about the longitudinal axis is realized, thereby moving the locking peg 63 via the first locking cam 38 and subsequently reaching the second groove 37. Subsequently, the locking peg 63 is moved along the one inner wall 37′, or along the opposite and correspondingly obliquely inclined inner wall 37″. As soon as the locking peg 63 is moved via the second locking cam 38′, the locking peg 63 is caught in an exactly defined end position in form-fitting engagement in semi-circular-shaped recess 39. Between the two locking cams 38 and 38′ positioned in circumferentially and at a distance to each other and the second groove 37, the locking peg 63 is guided between the two inner walls 37′ and 37″ with little clearance and freely movable.

As shown in FIG. 25, the second wall 37″ starting from the first locking cam 38 in direction of the semi-circular-shaped recess 39, is configured extending at an oblique angle relative to the first wall 37′. Between the two locking cams 38 and 38′ positioned at a distance to each other and disposed circumferentially at the adapter 30, the locking peg 63 is freely movable with little clearance in order to avoid any unwanted pinching between the two walls 37′ and 37″ disposed at a distance to each other and secured between the two locking cams 38 and 38′ against unintended separation.

Due to a rotating motion of the housing 10 in opposite direction and relative to the functional unit 50 (or reverse) the form-fitting connection can be separated and without further means and subsequently the functional unit 50 can be pulled in axial direction and separated from the cylindrical adapter 30 of the connecting member 25.

While the invention has been illustrated and described in connection with currently preferred embodiments shown and described in detail in FIG. 1 to FIG. 25, it is not intended to be limited to the details shown since various modifications and structural changes may be made without departing in any way from the spirit of the present invention. Essentially, the housing 10 is preferably configured as a hand piece, which can be form-fittingly connected by means of an axial sliding motion, and in the end position through a rotating sliding motion at about an 90° angle with the functional unit 50 without any additional means. In the end position, due to the manually or electrically operated drive means the first rod 12 supported in housing 10 is in operative connection with the sliding pin 60 disposed in the functional unit 50 for actuating the surgical tool 80 as schematically illustrated in FIGS. 1, 2, 10, 11, 15 and 22 connected to the distal end of the functional unit 50. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and their equivalents:

What is claimed is:

1. Device for removable connection of a functional unit provided with a manually or motorically operable surgical tool to a housing of an ophthalmologic instrument comprising:
a connecting member disposed in the housing, the connecting member connected to a rod, which is supported in the housing and movable in axial direction;
an adapter disposed at the connecting member configured for engagement with the functional unit; and
a sliding pin disposed in the functional unit and connected to the surgical tool; and
wherein a guide track is provided in one of the adapter or the functional unit for engagement into a form-fitting connection of the functional unit with the housing such that the rod when moved in axial direction by a drive can operatively engage with the sliding pin to thereby initiate actuation of the surgical tool.

2. The device of claim 1, wherein the guide track is conceived as a slotted guide track.

3. The device of claim 1, wherein the guide track is configured with a first groove extending in axial direction and a second groove extending substantially transverse to the first groove and bordered by a semi-circular recess, and wherein the functional unit is provided with a locking peg for traveling in the guide track such that after a rotational motion of the functional unit relative to the housing the locking peg is locked at the semicircular recess whereby the functional unit is form-fittingly connected to the housing.

4. The device of claim 3, wherein the second groove includes first and second inner walls extending circumferentially and at a distance from each other and wherein the first inner wall facing a front side is provided with two curved and arc-shaped locking cams oriented at a distance from each other with a curved part facing in the direction of the second inner wall.

5. The device of claim 4, wherein the first inner wall relative to the second wall facing the first wall is sloped in the direction of the semi-circular recess, so that the second groove starting from the first groove is tapered in direction of the recess.

6. The device of claim 4, wherein the first and second grooves are dimensioned for clearance of the locking peg when the locking peg is guided in the guide track in the direction of the recess and held by the second locking cam.

7. The device of claim 3, wherein the adapter disposed at the connecting member is cylindrical-shaped and starting from the front side has a length dimension of 6 mm and an outer diameter of 4.5 mm, and the first groove starting from the front side has a length of 4.5 mm and the second groove is positioned at an arced angle in the range from 75° to 120° relative to the first groove.

8. The device of claim 7, wherein the second groove is positioned with the arced angle of 90° relative to the first groove.

9. The device of claim 1, wherein the slot-shaped guide track is disposed in a recess of the functional unit and axially movable relative to the housing onto the adapter of the connecting member into one of an end position or a reverse position for respectively a form-fitting engagement or disengagement at the adapter, such that upon engagement, the first rod can be at the same time operatively engaged with the sliding pin and a second rod disposed in the functional unit for actuation of the surgical tool.

10. The device of claim 1, further comprising an intermediary member for threadedly receiving the functional unit and a locking peg, and disposed at the intermediary member for insertion into and locking engagement in the guide track in the adapter when the intermediary member is axially moved into an end position relative to the connecting member of the housing for a from-fitting connection to the adapter.

11. The device of claim 1, wherein the drive disposed in the housing is powered by a power source selected from the group consisting of electrical or hydraulic or pneumatic for providing an axial movement of the first rod to thereby actuate the surgical tool.

12. The device of claim 1, wherein the drive disposed in the housing is powered by a battery disposed in the housing.

13. The device of claim 1, wherein the connecting member includes a head piece connected to the adapter and at least one manually operable lever supported at the head piece and including a catch by which the head piece is brought into operative engagement with the axially movable first rod.

14. The device of claim 13, wherein the head piece is configured with slot-shaped recesses in circumferential arrangement, each of the recesses configured for supporting a lever when two or more levers are provided, and wherein each of the levers disposed with a catch held between two retaining pieces apart from each other at t distance is in operative engagement with the first rod.

15. The device of claim 1, wherein the housing split longitudinally into two elongate housing parts connected to each other at one end and movable relative to each other at another end includes a carrying arm disposed therebetween with the connecting member disposed at a distal end and with the first rod co-axially supported in the housing and connected to a drive mechanism, so that when the two housing parts are manually pressed together the first rod, which is movable in axial direction can be brought in operative engagement with the surgical tool which is form-fittingly connected to the functional unit disposed at the adapter.

16. The device of claim 15, wherein the first rod supported in the connecting piece is provided with a wedge-shaped sliding member for engagement with rollers supported at the housing, and wherein the two housing parts when pressed together can cause the first rod to slide in axial direction to thereby bring the first rod into engagement with the sliding pin and a second rod in operative engagement with the surgical tool which is form-fittingly connected to the functional unit disposed at the adapter.

17. The device of claim 15, wherein the carrying arm is provided with a longitudinally oriented recess in which the wedge-shaped sliding member can be guided in axial direction for fixed rotative engagement.

18. The device of claim 1, wherein the connecting member includes a guide sleeve disposed in the housing, a lever supported at the sleeve and the adapter for a form-fitting connection with the functional unit, and wherein the lever is provided with a catch, which is in operative engagement with the axially sliceable first rod for engagement with the sliding pin in operative engagement with the surgical tool which is form-fittingly connected to the functional unit disposed at the adapter.

19. The device of claim 18, wherein the guide sleeve is configured with an axial bore ending in a stop, and wherein the first rod includes a head piece for engagement with the catch of the lever such that upon movement of the lever the head piece can bear upon the stop.

20. The device of claim 1, wherein the housing, which is configured for receiving a drive mechanism of the type selected from the group of electric motor drive, hydraulic drive and pneumatic drive is conceived as a first subassembly and the functional unit which is provided with the surgical tool is conceived as a second subassembly, and wherein the two subassemblies are brought into operative engagement by a rotational motion relative to each other about the longitudinal axis for a form-fitting connection with the first rod and the sliding pin, and with each other.

21. The device of claim 1, wherein the housing is configured for receiving a battery for actuating a drive mechanism and is conceived as a first subassembly and the functional unit which is provided with the tool is conceived as a second subassembly, and wherein the two subassemblies are brought into operative engagement by a rotational motion relative to each other about the longitudinal axis for a form-fitting connection with the first rod and the sliding pin, and with each other.

22. The device of claim 1, further comprising at least one a lever provided at the housing for operative engagement with the axially movable first rod, and wherein the housing is configured as a first subassembly and the functional unit which is provided with the surgical tool is conceived as a second subassembly, and wherein the two subassemblies are brought into operative engagement by a rotational motion relative to each other about the longitudinal axis for a form-fitting connection with the first rod and the sliding pin, and with each other.

23. The device of claim 1, wherein the housing which forms a generally cylindrical hand piece and is split longitudinally into two elongate housing parts of substantially semi-circular configuration and the connecting member provided at the housing is conceived as a first subassembly and the functional unit which is provided with the surgical tool is conceived as a second subassembly, and wherein the two subassemblies are brought into operative engagement by a rotational motion relative to each other about the longitudinal axis for a form-fitting connection with the first rod and the sliding pin, and with each other.

24. The device of claim 1, wherein the housing including the connecting member is conceived as a first subassembly and the functional unit which is provided with the surgical tool and is threadedly connected to an intermediary member is conceived as a second subassembly, and wherein the two subassemblies are brought into operative engagement by a rotational motion relative to each other about the longitudinal axis for a form-fitting connection with the first rod and the sliding pin, and with each other.

25. The device of claim 1, wherein the housing including the connecting member and an intermediary member with an outer thread for form-fitting engagement with the connecting member is conceived as a first subassembly and the functional unit which is provided with an interior thread threadedly connected to the intermediary member and provided with the tool is conceived as a second subassembly, and wherein the two subassemblies are brought into operative engagement by a rotational motion relative to each other about the longitudinal axis for a form-fitting connection with the first rod and the sliding pin and with each other.

26. The device of claim 1, wherein the functional unit is provided with an inner thread is conceived as a first subassembly and the housing including the connecting member and the intermediary member provided with an outer thread for form-fitting threaded connection with the connecting member is conceived as second subassembly and wherein the two subassemblies are brought into operative engagement by a rotational motion relative to each other about the longitudinal axis for a form-fitting connection with the first rod and the sliding pin and with each other.
